(19) United States
(12) Patent Application Publication
(10) Pub. No.: US 2008/0078106 A1
(43) Pub. Date: Apr. 3, 2008

(54) Shoe for enhanced foot-to-ground tactile sensation and associated method

(76) Inventor: Donna Ilene Montgomery, Asheville, NC (US)

Correspondence Address: J. DEREL MONTEITH, JR.;CARTER, SCHNEDLER & MONTEITH, P.A., 56 CENTRAL AVENUE, SUITE 101, P.O. BOX 2985, ASHEVILLE, NC, 28802, US

(21) Appl. No.: 11/542,058

(22) Filed: Oct. 2, 2006

(51) Int. Cl. A43B 13/14; A61F 5/14

(52) U.S. Cl. 36/25 R; 36/153

(57) ABSTRACT

A shoe including a spikeless and cleatless sole configured to substantially conform to the contours of a surface engaged by the sole such that a human foot within the shoe conforms to the contours of the surface engaged by the sole substantially as if the foot were bare.

### BACKGROUND OF THE INVENTION

Athletes and sportsmen such as runners and golfers have long experimented with practicing their sports without wearing shoes. Some of these athletes and sportsmen experience better performance or at least a more pleasurable experience when they participate in their sports barefoot. For instance, some golfers experience better swing performance when barefoot due to the enhanced foot-to-ground tactile sensation that barefoot play provides. Playing barefoot enables them to sense and grip the contours of the ground surface more readily, thereby increasing balance and stability during a golf swing. However, golfers and other athletes and sportsmen who enjoy barefoot play are sometimes reluctant to go barefoot due to concerns about appearing too casual or informal or even foolish and silly.

### BRIEF DESCRIPTION OF THE DRAWINGS

FIG. 1 is a perspective view of a prior art shoe with spikes or cleats on its sole;

FIG. 2 is a perspective view of a shoe in accordance with an embodiment of the invention;

FIG. 3 is a cross-sectional side elevation view of the shoe shown in FIG. 2 while being worn on a human foot; and

FIG. 3a is a detail view of a portion of FIG. 3.

### DETAILED DESCRIPTION OF THE INVENTION

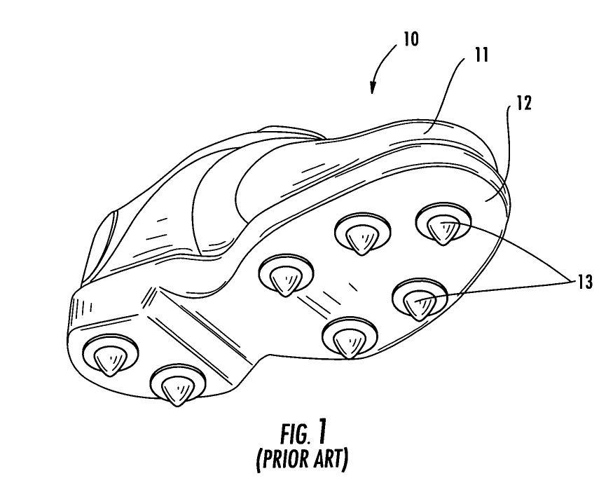

A prior art shoe is shown broadly in FIG. 1 at reference numeral 10. The shoe 10 includes an upper 11 attached to a sole 12. A plurality of spikes or cleats 13 are attached to or integral with the sole 12 of the shoe 10. When an athlete or sportsman such as a golfer wears a shoe similar to that shown in FIG. 1, the foot has little if any sensation of the detailed contours of a surface engaged by the shoe 10.

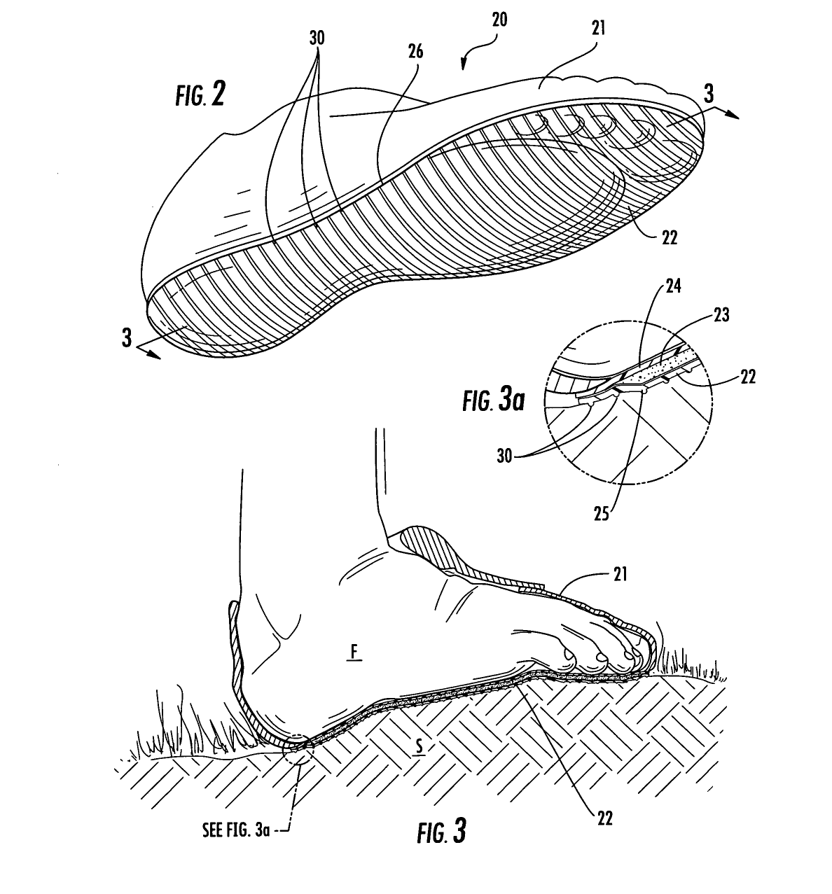

A shoe according to an embodiment of the invention is shown broadly in FIG. 2 at reference numeral 20, and in greater detail in FIGS. 3 and 3a. The shoe 20 includes an upper 21 attached to a sole 22 along a ridge 26 and may either be slipped or fastened onto a user's foot, as desired. The upper 21 is comprised of stretchable and breathable material such as nylon mesh. The sole 22 is devoid of spikes and cleats, but is formed or provided with ribs 30 to enhance traction between the shoe 20 and a surface “S” such as the ground engaged by the sole 22. During wear of the shoe 20, the sole 22 can flex both away from and toward the surface “S”, as desired. In order to help enhance the flexibility of the shoe 20, the ribs 30 do not extend to the periphery of the sole 22 and the sole 22 does not extend up and around the toe or heel portions of the shoe 20.

In the disclosed embodiments of the invention, when the shoe 20 has not been worn the sole 22 has a substantially uniform thickness between approximately three-eighths (⅜) inch and approximately one (1) inch and has a multiple-layer architecture (FIG. 3a) comprising a layer of gel 23 having a thickness between approximately one-quarter (¼) inch and one-half (½) inch bounded by an insole 24 of cloth, plastic, and/or rubber having a thickness between approximately one-sixteenth ( 1/16) inch and approximately one-quarter (¼) inch and an outsole 25 of cloth, plastic, and/or rubber having a thickness between approximately one-sixteenth ( 1/16) inch and approximately one-quarter (¼) inch. The layer of gel 23 is comprised of a highly flexible, jelly-like gel enclosed in a nylon or neoprene membrane (not shown). In the disclosed embodiments, the thickness of the ridge 26 is approximately one-eighth (⅛) inch or less so as not to hinder the flexing of the sole 22 during wear.

As shown in cross-section in FIGS. 3 and 3a, the sole 22 is thin and flexible. The sole 22 is configured to substantially conform to the contours of a surface “S” engaged by the sole 22 such that a human foot “F” within the shoe 20 conforms to the contours of the surface “S” engaged by the sole 22 substantially as if the foot “F” were bare. The sole 22 is further configured such that toes on the foot “F” may, as desired, grip the surface “S” engaged by the sole 22. Thus, the scope of the invention includes any sole material through which a foot “F” of a human wearer of the shoe 20 may substantially conform to, and, as desired, maintain a toe grip upon, the detailed contours of the surface “S” engaged by the shoe 20 substantially as if the foot “F” were bare.

Thus, a human foot “F” is enabled to wear a shoe 20, and to conform to the contours of a surface “S” engaged by the shoe 20 substantially as if the foot “F” were bare, by providing a shoe 20 in accordance with the invention and fitting and securing the shoe 20 onto the foot “F”.

A shoe for enhanced foot-to-ground tactile sensation and associated method is described above. Various details of the invention may be changed without departing from its scope. Furthermore, the foregoing description of embodiments of the invention and the best mode for practicing the invention are provided for the purpose of illustration only and not for the purpose of limitation—the invention being defined by the claims.

What is claimed is:

1. A shoe comprising a spikeless and cleatless sole configured to substantially conform to the contours of a surface engaged by the sole such that a human foot within the shoe conforms to the contours of the surface engaged by the sole substantially as if the foot were bare.

2. A shoe according to claim 1, wherein the sole comprises gel.

3. A shoe according to claim 1, wherein the sole is flexible.

4. A shoe according to claim 1, wherein the sole has a thickness between approximately three-eighths (⅜) inch and approximately one (1) inch.

5. A shoe according to claim 1, wherein the sole is further configured to enable toes on the foot to grip the surface engaged by the sole.

6. A shoe according to claim 1, wherein the surface is the ground.

7. A shoe, comprising:
an upper;
a spikeless and cleatless sole attached to the upper, the sole having a thickness between approximately three-eighths (⅜) inch and approximately one (1) inch.

8. A shoe according to claim 7, wherein the sole comprises gel.

9. A shoe according to claim 7, wherein the sole is flexible.

10. A shoe according to claim 7, wherein the sole is configured to substantially conform to the contours of a surface engaged by the sole such that a human foot within the shoe conforms to the contours of the surface engaged by the sole substantially as if the foot were bare.

11. A shoe according to claim 10, wherein the sole is further configured to enable toes on the foot to grip the surface engaged by the sole.

12. A method for enabling a human foot to wear a shoe and to conform to the contours of a surface engaged by the shoe substantially as if the foot were bare, said method comprising:
providing a shoe comprising a spikeless and cleatless sole configured to substantially conform to the contours of a surface engaged by the sole such that a human foot within the shoe conforms to the contours of the surface engaged by the sole substantially as if the foot were bare; and
fitting and securing the shoe onto the foot.

13. A method according to claim 12, wherein the sole of the shoe provided in said providing step comprises gel.

14. A method according to claim 12, wherein the sole of the shoe provided in said providing step is flexible.

15. A method according to claim 12, wherein the sole of the shoe provided in said providing step has a thickness between approximately three-eighths (⅜) inch and approximately one (1) inch.

16. A method according to claim 12, wherein the sole of the shoe provided in said providing step is further configured to enable toes on the foot to grip the surface engaged by the sole.

17. A method according to claim 12, wherein the surface is the ground.
